(19) United States
(12) Patent Application Publication
(10) Pub. No.: US 2007/0276425 A1
(43) Pub. Date: Nov. 29, 2007

(54) Painless Blood Sampling Lancet with Bundled Multiple Thin Needles

(76) Inventors: Stanley Kim, Upland, CA (US); Edward Kim, Upland, CA (US); Shyna Kim, Upland, CA (US); Audrey Kim, Upland, CA (US); Esther Kim, Upland, CA (US)

Correspondence Address: SHELDON MAK ROSE & ANDERSON PC, 100 East Corson Street, Third Floor, PASADENA, CA, 91103-3842, US

(21) Appl. No.: 11/420,767

(22) Filed: May 29, 2006

(51) Int. Cl. A61B 17/34

(52) U.S. Cl. 606/186

(57) ABSTRACT

A puncturing needle device has multiple (2 or more) thin needles which are mounted onto a body part or a syringe. The combination of thinness and multitude of needle can minimize pain while drawing or injecting sufficient amount of liquid such as blood or liquid drug. The needles can be different in their length.

### CROSS-REFERENCE TO RELATED APPLICATION

None.

### FEDERALLY SPONSORED RESEARCH

None.

### SEQUENCE LISTING

None.

### BACKGROUND

1. Field of Invention

This invention relates to a lancet device designed to minimize pain when it punctures the skin to obtain blood sample. A drop of blood is used for various blood tests, such as blood sugar testing for a diabetic patient. Presently, a typical lancet has single needle sized from 21-33 gauge. Because the thinner the puncturing needle, the less injury and pain, it is obvious that a thinner needle is desirable for blood sampling. However, the very thin needle may not be able to produce enough amount of blood necessary for testing. Therefore, we use multiple (2 or more) thin bundled needles, such as 34 gauged or thinner, so that the smaller amount of blood produced by each thin needle can coalesce to be a larger drop enough for testing. Because the needle is very thin, each needle elicits much less pain when puncturing the skin.

2. Prior Art

The lancet currently used for puncturing the skin to obtain blood sample has a single needle sized from 21-33 gauge in thickness and 3 mm in length. The lancet has the universal standard size of plastic body part sized 22 mm in length and 6 mm in diameter. The body part can be cocked into the different type of pen-shaped pricking device. Most of the pen-shaped skin pricking devices have a depth adjustment knob.

When deeper puncture is necessary to produce more amount of blood, these devices can make the needle penetrate the skin deeper. Although a very thin needle may cause less pain when puncturing skin, it may not produce enough blood for testing, and the patient will need to puncture the skin again only to cause more pain and discomfort.

### OBJECTS AND ADVANTAGES

The painless blood sampling lancet with bundled thin needles has unique features that enable it to minimize pain when puncturing the skin while producing enough amount of blood for testing. It is based on the fact that the pain caused by a single larger needle puncture is more than the pain by 2 separate half-sized thinner needles, while the amount of blood produced by the single larger needle would be the same as the sum of blood produced by 2 half-sized thinner needles.

1. The size of the needle is much thinner to effectively reduce pain when puncturing the skin.

2. A thinner needle is visually less intimidating.

3. In order to produce more blood, multiple (2 or more) needles need to be mounted on the plastic body part.

4. The length of each needle mounted on the plastic body part can be different.

This is because some patients have much more delicate and thinner skin that requires only a single puncture with a thin needle to obtain enough blood for testing. On the other hand, other patients have thicker skin which needs 2 or more simultaneous needle punctures for adequate blood sampling. By setting the depth adjustment knob of the commercially available skin pricking device, the patients can have a choice of one needle puncture or 2 or more simultaneous punctures. For example, let us assume that the lancet device has 2 needles mounted on the plastic body part with needle lengths of 1.5 mm and 2 mm respectively. By setting the depth adjustment knob to the deepest puncture level, both needles will puncture the skin producing more blood. If the depth of needle is set to penetrate shallow, then only 1 needle of the lancet device can puncture the skin producing less blood amount. However, for some patients with thin skin, only one needle puncture may be sufficient for blood sampling. In addition, certain laboratory tests may require less amount of blood.

5. This lancet device can be used for any commercially available skin pricking device because the needles are mounted on the universal standard sized plastic body part.

6. This invention can be applied for injecting or aspirating fluid when a hollow needle is used instead of non-hollow lancet. For example, a syringe can have 2 very thin needles instead of 1 thicker needle to inject liquid drug or aspirating body fluid. It causes less pain due to the thinness of needle when puncturing, but injects or draws more amount of body fluid with multiple needles.

### SUMMARY

The painless blood sampling lancet with bundled thin needles is a lancet device with the following features to minimize pain when puncturing the skin while producing enough blood for laboratory tests.

1. The size of the needle mounted on the plastic body part is thinner. The thinner the needle, the less the pain when puncturing the skin. A thinner needle is visually less intimidating.

2. Multiple (2 or more) needles are mounted on the body part to allow 2 or more simultaneous skin punctures producing adequate blood amount for laboratory testing.

4. The length of the needles mounted on the plastic body part can be different. By setting the depth adjustment knob of the commercially available skin pricking device to different levels, a patient can have either a single needle puncture or multiple simultaneous needle punctures.

4. This lancet device can be cocked into the various different types of pen-shaped pricking devices because of its standard universal size.

5. This invention can be applied for injecting or aspirating fluid when a hollow needle is used instead of non-hollow lancet. For example, a syringe can have 2 very thin needles instead of 1 thicker needle to inject liquid drug or aspirating body fluid. It causes less pain when puncturing, but injects or draws more body fluid with multiple needles.

### DRAWINGS

### Figures

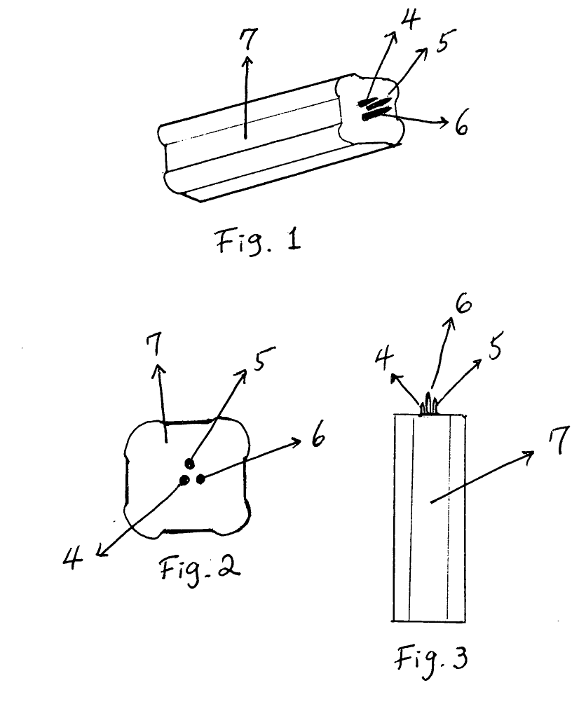

FIG. 1 is a perspective view of a painless blood sampling lancet with bundled multiple thin needles.

FIG. 2 is a frontal view of a painless blood sampling lancet with multiple thin needles.

FIG. 3 is a lateral view of a painless blood sampling lancet with multiple thin needles.

### REFERENCE NUMERALS

4. A thin needle with 36-gauge in diameter and 1 mm in length mounted on a plastic body part.

5. A thin needle with 36-gauge in diameter and 1.5 mm in length mounted on a plastic body part.

6. A thin needle with 36-gauge in diameter and 2 mm in length mounted on a plastic body part.

7. A plastic body part of a lancet device.

### DETAILED DESCRIPTION

FIG. 1 is a perspective view of a painless blood sampling lancet device with bundled multiple thin needles. In this device, 3 thin needles with the same 36 gauge but in different length (4,5,6) are mounted on a universal standard sized plastic body part (7). The size of the needles can be varied, such as 34 gauge, 35 gauge, 37 gauge, or higher gauges. The number of the needles mounted on the plastic body part can be two, or even four. The length of the needles mounted on the body part is 1 mm (4), 1.5 mm (5), and 2 mm (6) in this illustration. However, the lengths of the needles can be the same if multiple simultaneous skin punctures are desirable, but it gives no choice of a single needle puncture. The needle can be made by any materials hard enough to puncture the skin.

The plastic body part has the universal standard size with 22 mm in length and 6 mm in diameter. The size, shape and configuration of the plastic body part can be modified in order to fit the skin pricking device into which the lancet device is cocked. It can be made from any material such as plastics, or others, as long as it can be held and be cocked into the skin pricking device.

FIG. 2 is a frontal view of the lancet device. Three thin needles (4,5,6) are mounted on a body part (7).

FIG. 3 is a lateral view of the lancet device. Three thin needles with different length (4,5,6) are mounted on the body part (7).

Although the description above contains many specificities, these should not be construed as limiting the scope of the invention but as merely providing illustrations of some of the presently preferred embodiments of this invention. For example, when a patient receives an injection of liquid medicine, 2 thinner hollow needles mounted on a syringe rather than 1 thicker needle can cause less pain when puncturing the skin, while delivering sufficient amount of the medicine. Thus, the scope of this invention should be determined by the appended claims and their legal equivalents, rather than by the examples given.

### OPERATION

A painless blood sampling lancet with bundled multiple thin needles can be used either manually by hand, or mechanically by a pricking device. When used manually, the plastic body part (7) is held with the fingers and the needles are poked in the skin. When used mechanically, the lancet device is cocked into a commercially available skin pricking device. By releasing the trigger of the pricking device the needles hit and puncture the skin. The desirable samples can be obtained by gently squeezing the pricked skin. The area of the skin poked can be the finger, palm, heel, foot, earlobe, or any part of the body where the blood sample can be obtained.

1. A system for obtaining a blood sample from an individual comprising:
a lancet having a plurality of needles mounted onto a lancet body; and
a lancing device configured to retain the lancet body of the lancet and to deliver the needles to a skin surface of the individual so as to puncture the skin surface.

2. The system of claim 1, wherein each of the plurality of needles mounted onto the lancet body has a length which is equal.

3. The system of claim 1, wherein at least one of the plurality of needles projects from the surface of the lancet body by a different distance compared to the remainder of the plurality of needles.

4. The system of claim 1, wherein each of the plurality of needles of the lancet has a diameter which is at least 34 gauge or smaller.

5. The system of claim 4, wherein each of the plurality of needles of the lancet has a diameter selected from the group consisting of 34 gauge, 35 gauge, 36 gauge, and 37 gauge.

6. The system of claim 1, wherein each of the plurality of needles of the lancet is a solid needle.

7. The system of claim 1, wherein the plurality of needles are mounted perpendicularly with respect to a surface of the lancet body.

8. The system of claim 1, wherein the lancing device comprises a depth adjustment control.

9. A method of obtaining a blood sample from an individual, comprising:
providing a lancet, the lancet comprising a lancet body and a plurality of needles mounted on the lancet body;
puncturing a skin surface of the individual with the plurality of needles; and
collecting blood from the skin surface of the individual, thereby obtaining the blood sample.

10. The method of claim 9, wherein at least one of the plurality of needles projects from the lancet body by a different distance compared to the remainder of the plurality of needles.

11. The method of claim 9, wherein the skin surface is located on a body part selected from the group consisting of a finger, a palm, a heel, and an earlobe.

12. The method of claim 9, wherein the lancet is delivered by a lancing device.

13. The method of claim 9, wherein the plurality of needles are solid.

14. The method of claim 9, wherein the plurality of needles are at least 34 gauge needles.

15. The method of claim 14, wherein each of the plurality of needles of the lancet has a diameter selected from the group consisting of 34 gauge, 35 gauge, 36 gauge, and 37 gauge.

16. The method of claim 9, further comprising the step of performing a blood sugar test with the blood sample.
